(19) United States
(12) Patent Application Publication
(10) Pub. No.: US 2023/0218740 A1
(43) Pub. Date: Jul. 13, 2023

(54) CORONAVIRUS VACCINES COMPRISING A TLR9 AGONIST

(71) Applicant: Dynavax Technologies Corporation, Emeryville, CA (US)

(72) Inventors: John D. CAMPBELL, San Francisco, CA (US); Robert S. JANSSEN, Palm Springs, CA (US); David NOVACK, Oakland, CA (US)

(73) Assignee: Dynavax Technologies Corporation, Emeryville, CA (US)

(21) Appl. No.: 17/908,221

(22) PCT Filed: Mar. 1, 2021
(86) PCT No.: PCT/US2021/020318

§ 371 Date: Aug. 30, 2022

Related U.S. Application Data
(60) Provisional application No. 62/983,737, filed on Mar. 1, 2020.

(51) Int. Cl. A61K 39/215; A61K 39/39

(52) U.S. Cl. CPC A61K 39/215; A61K 39/39; A61K 2039/55561

(57) ABSTRACT

The present disclosure relates to immunogenic compositions comprising a severe acute respiratory syndrome coron-avirus 2 (SARS-CoV-2) antigen, and a toll-like receptor 9 (TLR9) agonist, such as an oligonucleotide comprising an unmethylated cytidine-phospho-guanosine (CpG) motif. The immunogenic compositions are suitable for stimulating an immune response against a SARS-CoV-2 in an individual in need thereof.

### CROSS-REFERENCE TO RELATED APPLICATIONS

This application claims priority to and the benefit of U.S. Provisional Application No. 62/983,737, filed Mar. 1, 2020, the disclosure of which is incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 377882007340SEQLIST.TXT, date recorded: Feb. 26, 2021, size: 12 KB).

### FIELD

The present disclosure relates to immunogenic compositions comprising a severe acute respiratory syndrome coronavirus 2 (SARS-CoV-2) antigen, and a toll-like receptor 9 (TLR9) agonist, such as an oligonucleotide comprising an unmethylated cytidine-phospho-guanosine (CpG) motif. The immunogenic compositions are suitable for stimulating an immune response against a SARS-CoV-2 in an individual in need thereof.

### BACKGROUND

Coronavirus disease 2019 (COVID-19) is caused by severe acute respiratory syndrome coronavirus 2 (SARS-CoV-2). Initial symptoms of COVID-19, also known as Wuhan pneumonia, include one or more of fever, cough, and shortness of breath appearing within about 2-14 days of exposure to SARS-CoV-2. Although most cases of COVID-10 are mild, nearly 5% progress to respiratory failure, septic shock and/or multiple organ failure, with a case fatality rate of about 2.3% (Wu and McGoogan, JAMA, 323(13):1239-1242, 2020).

SARS-CoV-2 is spread through contact with respiratory droplets produced when an infected person coughs or exhales. According to the World Health Organization (WHO), as of Mar. 1, 2020 there are over 85,000 confirmed COVID-19 cases in 60 countries leading WHO to declare the current outbreak as a public health emergency of international concern. According to the worldometer, nearly one year later there are over 110 million coronavirus cases accounting for over 2.5 million deaths worldwide, with over 29 million coronaviruses cases accounting for over 500,000 deaths in the United States alone. In order to prevent person-to-person transmission of SARS-CoV-2, basic measures such as frequently washing hands, avoidance of touching eyes, nose and mouth, and an avoiding travel and public activities are recommended.

However, to reduce the risk of SARS-CoV-2 infection without curtailing everyday activities, a COVID-19 vaccine is needed. In particular, a COVID-19 vaccine that is able to rapidly induce an immune response against SARS-CoV-2 is urgently needed.

### SUMMARY

The present disclosure relates to immunogenic compositions comprising a severe acute respiratory syndrome coronavirus 2 (SARS-CoV-2) antigen, and a toll-like receptor 9 (TLR9) agonist, such as an oligonucleotide comprising an unmethylated cytidine-phospho-guanosine (CpG) motif. The immunogenic compositions are suitable for stimulating an immune response against a SARS-CoV-2 in an individual in need thereof.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., about 3000 µg of CpG 1018 refers to 2700 µg to 3300 µg of CpG 1018).

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. A nucleotide is a phosphate ester of a nucleoside.

The terms “CpG”, “CpG motif,” and “cytosine-phosphate-guanosine,” as used herein, refer to an unmethylated cytidine-phospho-guanosine dinucleotide, which when present in an oligonucleotide contributes to a measurable immune response in vitro, in vivo and/or ex vivo. Examples of measurable immune responses include, but are not limited to, antigen-specific antibody production, secretion of cytokines, activation or expansion of lymphocyte populations, such as NK cells, CD4+ T lymphocytes, CD8+ T lymphocytes, B lymphocytes, and the like. Preferably, the CpG oligonucleotide preferentially activates a Th1-type response.

An “effective amount” or a “sufficient amount” of a substance is that amount sufficient to effect beneficial or desired results, including clinical results, and, as such, an “effective amount” depends upon the context in which it is being applied. In the context of administering an immunogenic composition, an effective amount contains sufficient antigen and TLR9 agonist to stimulate an immune response (preferably a seroprotective level of antibody to the antigen).

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 5-fold to 500-fold or over, or from 5, 10, 50, or 100-fold to 500, 1,000, 5,000, or 10,000-fold.

As used herein the term “immunization” refers to a process that increases a mammalian subject’s reaction to antigen and therefore improves its ability to resist or overcome infection.

The term “vaccination” as used herein refers to the introduction of vaccine into a body of a mammalian subject.

“Adjuvant” refers to a substance which, when added to a composition comprising an antigen, nonspecifically enhances or potentiates an immune response to the antigen in the recipient upon exposure.

### DETAILED DESCRIPTION

The present disclosure relates to immunogenic compositions comprising a severe acute respiratory syndrome coronavirus 2 (SARS-CoV-2) antigen and a toll-like receptor 9 (TLR9) agonist, such as an oligonucleotide comprising an unmethylated cytidine-phospho-guanosine (CpG) motif. The immunogenic compositions are suitable for stimulating an immune response against a SARS-CoV-2 in an individual in need thereof.

### I. Immunogenic Compositions and Kits

The present disclosure relates to immunogenic compositions for stimulating an immune response against a severe acute respiratory syndrome coronavirus 2 (SARS-CoV-2), comprising a SARS-CoV-2 antigen and a toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, and the SARS-CoV-2 antigen and the oligonucleotide are present in the immunogenic composition in amounts effective to stimulate an immune response against the SARS-CoV-2 antigen in a mammalian subject, such as a human subject in need thereof.

### A. Toll-Like Receptor 9 (TLR9) Agonists

Toll-like receptors (TLRs) are expressed on dendritic cells and other innate immune cells and are among the most important receptors for stimulating a response to the presence of invading pathogens. Humans have multiple types of TLRs that are similar in structure but recognize different parts of viruses or bacteria. By activating specific TLRs, it is possible to stimulate and control specific types of innate immune responses that can be harnessed to enhance adaptive responses.

TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010). Based on structure and biological function, CpG-ODNs have been divided into three general classes: CpG-A, CpG-B, and CpG-C (Campbell, Methods Mol Biol, 1494:15-27, 2017). The degree of B cell activation varies between the classes with CpG-A ODNs being weak, CpG-C ODNs being good, and CpG-B ODNs being strong B cell activators. Oligonucleotide TLR9 agonists of the present disclosure are preferably good B cell activators (CpG-C ODN) or more preferably strong (CpG-B ODN) B cell activators.

Optimal oligonucleotide TLR9 agonists often contain a palindromic sequence following the general formula of: 5′-purine-purine-CG-pyrimidine-pyrimidine-3′, or 5′-purine-purine-CG-pyrimidine-pyrimidine-CG-3′ (U.S. Pat. No. 6,589,940). TLR9 agonism is also observed with certain non-palindromic CpG-enriched phosphorothioate oligonucleotides, but may be affected by changes in the nucleotide sequence. Additionally, TLR9 agonism is abolished by methylation of the cytosine within the CpG dinucleotide. Accordingly in some embodiments, the TLR9 agonist is an oligonucleotide of from 8 to 35 nucleotides in length comprising the sequence 5′-AACGTTCG-3′. In some embodiments, the oligonucleotide is greater than 8, 9, 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, the TLR9 agonist is an oligonucleotide of from 10 to 35 nucleotides in length comprising the sequence 5′-AACGTTCGAG-3′ (SEQ ID NO:3). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length.

Researchers at Dynavax Technologies Corporation (Emeryville, CA) have identified a 22-mer phosphorothioate linked oligodeoxynucleotide, CpG 1018, which contains specific sequences that can substantially enhance the immune response to co-administered antigens across species (Campbell, Methods Mol Biol, 1494:15-27, 2017). CpG 1018 (5′-TGACTGTGAA CGTTCGAGAT GA-3′, set forth as SEQ ID NO:1) was chosen after screening a broad panel of oligonucleotides for immunostimulatory activity in vitro and in vivo. CpG 1018 is a CpG-B ODN that is active in mice, rabbits, dogs, baboons, cynomolgus monkeys, and humans. Thus in some preferred embodiments, the TLR9 agonist is an oligonucleotide comprising the sequence of SEQ ID NO:1.

Although the exemplary oligonucleotide TLR9 agonist, CpG 1018, is a CpG-ODN, the present disclosure is not restricted to fully DNA molecules. That is, in some embodiments, the TLR9 agonist is a DNA/RNA chimeric molecule in which the CpG(s) and the palindromic sequence are deoxyribonucleic acids and one or more nucleic acids outside of these regions are ribonucleic acids. In some embodiments, the CpG oligonucleotide is linear. In other embodiments, the CpG oligonucleotide is circular or includes hairpin loop(s). The CpG oligonucleotide may be single stranded or double stranded.

In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine. In some embodiments, the TLR9 agonist is an oligonucleotide comprising the sequence 5′-TCG₁AACG₁TTCG₁-3′ (SEQ ID NO:2), in which G₁ is 2′-deoxy-7-deazaguanosine. In some embodiments, the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

A unit dose of the immunogenic composition, which is typically a 0.5 ml dose, may comprises from about 500 µg to about 5000 µg of the CpG oligonucleotide, preferably from about 750 µg to about 3000 µg of the CpG oligonucleotide. In some embodiments, a 0.5 ml dose of the immunogenic composition comprises greater than about 500, 750, 1000, or 1250 µg of the CpG oligonucleotide, and less than about 3250, 3000, 2750, 2500, 2250, 2000, or 1750 µg of the CpG oligonucleotide. In some embodiments, a 0.5 ml dose of the immunogenic composition comprises about 750, 1500, or 3000 µg of the CpG oligonucleotide. In some embodiments, a 0.5 ml dose of the immunogenic composition comprises about 250 µg of the CpG oligonucleotide. In some embodiments, a 0.5 ml dose of the immunogenic composition comprises about 500 µg of the CpG oligonucleotide. In some embodiments, a 0.5 ml dose of the immunogenic composition comprises about 750 µg of the CpG oligonucleotide. In some embodiments, a 0.5 ml dose of the immunogenic composition comprises about 1000 µg of the CpG oligonucleotide. In some embodiments, a 0.5 ml dose of the immunogenic composition comprises about 1500 µg of the CpG oligonucleotide. In some embodiments, a 0.5 ml dose of the immunogenic composition comprises about 3000 µg of the CpG oligonucleotide.

The CpG oligonucleotides described herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In some embodiment, the CpG oligonucleotides are in the ammonium, sodium, lithium, or potassium salt form. In one preferred embodiment, the CpG oligonucleotides are in the sodium salt form.

### B. SARS-CoV-2 Antigens

A SARS-CoV-2 antigen of the immunogenic compositions of the present disclosure comprises at least one SARS-CoV-2 protein or fragment thereof. In preferred embodiments, the SARS-CoV-2 antigen is recognized by SARS-CoV-2 reactive antibodies and/or T cells. In some embodiments, the SARS-CoV-2 antigen is an inactivated whole virus (COVID-19 virus). In other embodiments, the SARS-CoV-2 antigen is a subunit of the virus. In some embodiments, the SARS-CoV-2 antigen comprises a structural protein of SARS-CoV-2 or a fragment thereof. In some embodiments, the structural protein of SARS-CoV-2 comprises one or more of the group consisting of the spike (S) protein, the membrane (M) protein, nucleocapsid (N) protein, and envelope (E) protein. In some embodiments, the SARS-CoV-2 antigen comprises or further comprises a non-structural protein of SARS-CoV-2 or a fragment thereof. The nucleotide sequence of a representative SARS-CoV-2 isolate (Wuhan-Hu-1) is set forth as GenBank No. MN908947.3 (Wu et al., Nature, 579:265-269, 2020).

The amino acid sequence of a SARS-CoV-2 S protein is set forth as SEQ ID NO:4:

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT. The signal peptide extend
s from residues 1-13, the extracellular region

extends from residues 14-1213, the transmembrane domain extends from residues 1214-1236, and the cytoplasmic domain extends from residues 1237-1273.

In some preferred embodiments, the SARS-CoV-2 antigen comprises the receptor-binding domain (RBD) of the S protein, which is set forth as SEQ ID NO:5:

NSNNLDSKVGGNYNYLYRLFRKSNLKPFERDISTEIYQAGSTPCNGVEGF
NCYFPLQSYGFQPTNGVGYQPYR.

In some embodiments, the SARS-CoV-2 antigen comprises a variant of the RBD of the S protein having an amino acid sequence that it at least 75%, 85%, 90%, 95%, 96%, 97%, 98% or 99% identical to SEQ ID NO:5. In some preferred embodiments, the SARS-CoV-2 antigen comprises the extracellular region of the S protein extending from residues 14-1213 of SEQ ID NO:4, or an amino acid sequence that it at least 75%, 85%, 90%, 95%, 96%, 97%, 98% or 99% identical to residues 14-1213 of SEQ ID NO:4. That is, in some embodiments, the SARS-CoV-2 antigen comprises a truncated, S protein devoid of signal peptide, transmembrane and cytoplasmic domains of a full length S protein. In some embodiments, the SARS-CoV-2 antigen is a recombinant protein, while in other embodiments, the SARS-CoV-2 antigen is purified from virions. In some preferred embodiments, the SARS-CoV-2 antigen is an isolated antigen.

A unit dose of the immunogenic composition, which is typically a 0.5 ml dose, may comprise from about 10 µg to about 100 µg of the SARS-CoV-2 antigen, preferably from about 25 µg to about 75 µg of the SARS-CoV-2 antigen, preferably from about 40 µg to about 60 µg of the SARS-CoV-2 antigen, or about 50 µg of the SARS-CoV-2 antigen. In some embodiments, the dose contains from about 3 µg to about 30 µg of the SARS-CoV-2 antigen, or about 3 µg, or about 9 µg, or about 30 µg of the SARS-CoV-2 antigen. In some embodiments, the dose contains from about 5 µg to about 25 µg of the SARS-CoV-2 antigen, or about 5 µg, or about 15 µg, or about 25 µg of the SARS-CoV-2 antigen. In some embodiments, the dose contains from about 0.25 µg to about 25 µg of the SARS-CoV-2 S antigen. In some embodiments, the dose contains from about 15 µg to about 50 µg of the SARS-CoV-2 antigen, or about 15 µg, or about 25 µg, or about 50 µg of the SARS-CoV-2 antigen.

### C. Additional Components

The immunogenic compositions of the present disclosure may comprise one or more additional components, such as one or more excipients, another adjuvant, and/or additional antigens.

### 1. Excipients

Pharmaceutically acceptable excipients of the present disclosure include for instance, solvents, bulking agents, buffering agents, tonicity adjusting agents, and preservatives (Pramanick et al., Pharma Times, 45:65-77, 2013). In some embodiments the immunogenic compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the immunogenic compositions comprise an aqueous vehicle as a solvent. Suitable vehicles include for instance sterile water, saline solution, phosphate buffered saline, and Ringer’s solution. In some embodiments, the composition is isotonic.

The immunogenic compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The immunogenic compositions may comprise a tonicity adjusting agent. Suitable tonicity adjusting agents include for instance dextrose, glycerol, sodium chloride, glycerin and mannitol.

The immunogenic compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The immunogenic compositions may comprise a preservative. Suitable preservatives include for instance antioxidants and antimicrobial agents. However, in preferred embodiments, the immunogenic composition is prepared under sterile conditions and is in a single use container, and thus does not necessitate inclusion of a preservative.

### 2. Additional Adjuvants

Adjuvants are known in the art and include, but are not limited to, alum (aluminum salts), oil-in-water emulsions, water-in-oil emulsions, liposomes, and microparticles, such as poly(lactide-co-glycolide) microparticles (Shah et al., Methods Mol Biol, 1494:1-14, 2017). In some embodiments, the immunogenic compositions further comprises an aluminum salt adjuvant to which the SARS-CoV-2 antigen is adsorbed. In some embodiments, the aluminum salt adjuvant comprises one or more of the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant consists of aluminum hydroxide. In some embodiments, a unit dose of the immunogenic composition, which is typically a 0.5 ml dose, comprises from about 0.25 to about 0.50 mg Al³⁺, or about 0.35 mg Al³⁺, or about 0.375 mg Al³⁺. In some embodiments, a 0.5 ml unit dose of the immunogenic composition comprises from about 0.05 to about 0.25 mg Al³⁺. In some embodiments, a 0.5 ml dose of the immunogenic composition comprises greater than about 0.050, 0.075, 0.100, 0.125, 0.150, 0.175, 0.200, 0.225, or 0.250 mg Al³⁺, and less than about 0.50, 0.45, 0.40, 0.35, 0.30 or 0.25 mg Al³⁺, provided that the minimum is lower than the maximum.

In other embodiments, the immunogenic composition further comprises an additional adjuvant. Other suitable adjuvants include, but are not limited to, squalene-in-water emulsion (e.g., MF59 or AS03), TLR3 agonists (e.g., poly-IC or poly-ICLC), TLR4 agonists (e.g., bacterial lipopolysaccharide derivatives such monophosphoryl lipid A (MPL), and/or a saponin such as Quil A or QS-21, as in AS01 or AS02), a TLR5 agonist (bacterial flagellin), and TLR7 and/or TLR8 agonists (imidazoquinoline derivatives such as imiquimod, and resiquimod)(Coffman et al., Immunity, 33:492-503, 2010). In some embodiments, the additional adjuvant comprises MPL and alum (e.g., AS04). For veterinary use and for production of antibodies in non-human animals, mitogenic components of Freund’s adjuvant (both complete and incomplete) can be used.

### D. Kits

The present disclosure also provides kits comprising: i) an immunogenic composition comprising a SARS-CoV-2 antigen and a toll-like receptor 9 (TLR9) agonist, such as a CpG oligonucleotide; and ii) a set of instructions for administration of the immunogenic composition to stimulate an immune response against the SARS-CoV-2 antigen in a mammalian subject, such as a human subject in need thereof. Additionally, the present disclosure provides kits comprising: i) a first composition comprising a SARS-CoV-2 antigen; ii) a second composition comprising a TLR9 agonist, such as a CpG oligonucleotide; iii) instructions for mixing the first composition with the second composition to prepare an immunogenic composition; and optionally iv) a further set of instructions for administration of the immunogenic composition to stimulate an immune response against the SARS-CoV-2 antigen in a mammalian subject, such as a human subject in need thereof. In some embodiments, the CpG oligonucleotide comprises the sequence 5′-AACGTTCG-3′. In some embodiments, the CpG oligonucleotide comprises the sequence 5′-AACGTTCGAG-3′ (SEQ ID NO:3). In some preferred embodiments, the CpG oligonucleotide comprises the sequence of 5′-TGACTGTGAA CGTTCGAGAT GA-3′ (SEQ ID NO: 1).

The kits may comprise an immunogenic composition packaged appropriately. For example, if the immunogenic composition is a freeze-dried power, a vial with a resilient stopper is normally used so that the powder may be easily resuspended by injecting fluid (e.g., sterile water, saline, etc.) through the resilient stopper. In some embodiments, the kits comprise a device for administration (e.g., syringe and needle for intramuscular injection). The instructions relating to the use of the immunogenic composition generally include information as to dosage, schedule and route of administration for the intended methods of use.

### II. Methods Of Use

The present disclosure relates to methods for stimulating an immune responses against a severe acute respiratory syndrome coronavirus 2 (SARS-CoV-2), comprising: administering an immunogenic composition comprising a SARS-CoV-2 antigen and a toll-like receptor 9 (TLR9) agonist, such as a CpG oligonucleotide, to a mammalian subject so as to stimulate an immune response against the SARS-CoV-2 antigen in the mammalian subject. The immunogenic compositions of the present disclosure are intended for active immunization against COVID-19. In preferred embodiments, the immunogenic compositions are to be administered by intramuscular injection, optionally in a volume of about 0.5 mL (e.g., unit dose). In some embodiments, the intramuscular injection is into the deltoid muscle of the upper arm of a human subject in need thereof. In some embodiments, one dose of the immunogenic composition is administered. In other embodiments, a first dose and a second dose of the immunogenic composition are administered, with the second dose administered from about 2 weeks to 8 weeks after the first dose, or from about 3 weeks to 6 weeks after the first dose, or about 4 weeks after the first dose. In some preferred embodiments, the second dose is administered about 3 or 4 weeks after the first dose. In some embodiments, the second dose is administered about 3 weeks after the first dose. In some embodiments, the second dose is administered about 4 weeks after the first dose.

“Stimulating” an immune response, means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response includes but is not limited to one or more of the group consisting of: stimulating cytokine production; stimulating B lymphocyte proliferation; stimulating antibody production; stimulating interferon pathway-associated gene expression; stimulating chemoattractant-associated gene expression; and stimulating plasmacytoid dendritic cell (pDC) maturation. In some preferred embodiments, stimulating an immune response comprises increasing an antigen-specific antibody response in the subject.

### ENUMERATED EMBODIMENTS

Embodiment 1. An immunogenic composition for stimulating an immune response against a severe acute respiratory syndrome coronavirus 2 (SARS-CoV-2), comprising a SARS-CoV-2 antigen and a toll-like receptor 9 (TLR9) agonist, wherein the TLR9 agonist is an oligonucleotide of from 10 to 35 nucleotides in length comprising an unmethylated cytidine-phospho-guanosine (CpG) motif, and the SARS-CoV-2 antigen and the oligonucleotide are present in the immunogenic composition in amounts effective to stimulate an immune response against the SARS-CoV-2 antigen in a mammalian subject.

Embodiment 2. The composition of embodiment 1, wherein the oligonucleotide comprises the sequence 5′-AACGTTCGAG-3′ (SEQ ID NO:3).

Embodiment 3. The composition of embodiment 1, wherein the oligonucleotide comprises the sequence of 5′-TGACTGTGAA CGTTCGAGAT GA-3′ (SEQ ID NO: 1).

Embodiment 4. The composition of any one of embodiments 1-3, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine.

Embodiment 5. The composition of embodiment 4, wherein the oligonucleotide comprises the sequence 5′-TCG₁AACG₁TTCG₁-3′ (SEQ ID NO:2) in which G₁ is 2′-deoxy-7-deazaguanosine, optionally wherein the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

Embodiment 6. The composition of any one of embodiments 1-5, wherein the oligonucleotide comprises at least one phosphorothioate linkage, or wherein all nucleotide linkages are phosphorothioate linkages.

Embodiment 7. The composition of any one of embodiments 1-6, wherein the oligonucleotide is a single-stranded oligodeoxynucleotide.

Embodiment 8. The composition of any one of embodiments 1-7, wherein a 0.5 ml dose of the immunogenic composition comprises from about 750 to about 3000 µg of the oligonucleotide, or wherein the immunogenic composition comprises about 750 µg, about 1000 µg, about 1500 µg, or about 3000 µg of the oligonucleotide.

Embodiment 9. The composition of any one of embodiments 1-8, wherein the SARS-CoV-2 antigen is an inactivated whole SARS-CoV-2.

Embodiment 10. The composition of embodiment 9, wherein the SARS-CoV-2 is inactivated by treatment with one or both of formalin and ultraviolet light.

Embodiment 11. The composition of any one of embodiments 1-8, wherein the SARS-CoV-2 antigen comprises the receptor-binding domain (RBD) of the SARS-CoV-2 spike (S) protein.

Embodiment 12. The composition of embodiment 11, wherein the SARS-CoV-2 antigen comprises a truncated, recombinant S protein devoid of signal peptide, transmembrane and cytoplasmic domains of a full length S protein.

Embodiment 13. The composition of embodiment 11 or 12, wherein the SARS-CoV-2 antigen further comprises one or more of the SARS-CoV-2 membrane (M) protein, nucleocapsid (N) protein, and envelope (E) protein.

Embodiment 14. The composition of any one of embodiments 1-13, further comprising an aluminum salt adjuvant.

Embodiment 15. The composition of embodiment 14, wherein the aluminum salt adjuvant comprises one or more of the group consisting of amorphous aluminum hydroxyphosphate sulfate, aluminum hydroxide, aluminum phosphate, and potassium aluminum sulfate

Embodiment 16. The composition of embodiment 14, wherein the aluminum salt adjuvant comprises aluminum hydroxide.

Embodiment 17. The composition of any one of embodiments 14-16, wherein a 0.5 ml dose of the immunogenic composition comprises from about 0.25 to about 0.50 mg Al³⁺, or about 0.250 mg Al³⁺, or about 0.375 mg Al³⁺, or about 0.05 to about 0.50 mg Al³⁺, or about 0.075 to about 0.175 mg Al³⁺.

Embodiment 18. The composition of any one of embodiments 1-17, wherein the mammalian subject is a human subject.

Embodiment 19. A kit comprising:
i) the immunogenic composition of any one of embodiments 1-18, and
ii) instructions for administration of the composition to stimulate an immune response against the SARS-CoV-2 antigen in the mammalian subject.

Embodiment 20. The kit of embodiment 19, further comprising iii) a syringe and needle for intramuscular injection of the immunogenic composition.

Embodiment 21. A method for stimulating an immune response against a severe acute respiratory syndrome coronavirus 2 (SARS-CoV-2) in a mammalian subject, comprising administering the immunogenic composition of any one of embodiments 1-18 to a mammalian subject in an amount effective to stimulate an immune response against the SARS-CoV-2 antigen in the mammalian subject.

Embodiment 22. The method of embodiment 21, wherein the mammalian subject is a human subject and/or the immunogenic composition is administered by intramuscular injection.

Embodiment 23. Use of the immunogenic composition of any one of embodiments 1-18 for stimulating an immune response against a severe acute respiratory syndrome coronavirus 2 (SARS-CoV-2) in a mammalian subject, the method comprising administering to the subject an effective amount of the immunogenic composition.

Embodiment 24. Use of the immunogenic composition of any one of embodiments 1-18 for protecting a mammalian subject from infection with severe acute respiratory syndrome coronavirus 2 (SARS-CoV-2), the method comprising administering to the subject an effective amount of the immunogenic composition.

Embodiment 25. Use of the immunogenic composition of any one of embodiments 1-18 for preventing a mammalian subject from contracting COVID-19 disease, the method comprising administering to the subject an effective amount of the immunogenic composition.

Embodiment 26. The use of any one of embodiments 23-25, wherein the mammalian subject is a human subject and/or the immunogenic composition is administered by intramuscular injection.

We claim:

1. An immunogenic composition for stimulating an immune response against a severe acute respiratory syndrome coronavirus 2 (SARS-CoV-2), comprising a SARS-CoV-2 antigen and a toll-like receptor 9 (TLR9) agonist, wherein the TLR9 agonist is an oligonucleotide of from 10 to 35 nucleotides in length comprising an unmethylated cytidine-phospho-guanosine (CpG) motif, and the SARS-CoV-2 antigen and the oligonucleotide are present in the immunogenic composition in amounts effective to stimulate an immune response against the SARS-CoV-2 antigen in a mammalian subject.

2. The composition of claim 1, wherein the oligonucleotide comprises the sequence
5’-AACGTTCGAG-3’
(SEQ ID NO:3).

3. The composition of claim 1, wherein the oligonucleotide comprises the sequence of
5’-TGACTGTGAA CGTTCGAGAT GA-3’
(SEQ ID NO: 1).

4. The composition of claim 1, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2’substituted-arabinoguanosine, and 2′-O-substituted-arabinoguanosine.

5. The composition of claim 4, wherein the oligonucleotide comprises the sequence
5’-TCG₁AACG₁TTCG₁-3’
(SEQ ID NO:2) in which G₁ is 2′-deoxy-7-deazaguanosine, optionally wherein the oligonucleotide comprises the sequence
5′-TCG₁AACG₁TTCG₁-X-G₁CTTG₁CAAG₁CT-5′,
and in which G₁ is 2′-deoxy-7-deazaguanosine and X is glycerol (5′-SEQ ID NO:2-3′-X-3′-SEQ ID NO:2-5′).

6. The composition of claim 3, wherein the oligonucleotide comprises at least one phosphorothioate linkage, or wherein all nucleotide linkages are phosphorothioate linkages.

7. The composition of claim 6, wherein the oligonucleotide is a single-stranded oligodeoxynucleotide.

8. The composition of claim 7, wherein a 0.5 ml dose of the immunogenic composition comprises from about 750 to about 3000 µg of the oligonucleotide, or wherein the immunogenic composition comprises about 750 µg, about 1500 µg, or about 3000 µg of the oligonucleotide.

9. The composition of claim 8, wherein the SARS-CoV-2 antigen is an inactivated whole SARS-CoV-2.

10. The composition of claim 9, wherein the SARS-CoV-2 is inactivated by treatment with one or both of formalin and ultraviolet light.

11. The composition of claim 8, wherein the SARS-CoV-2 antigen comprises the receptor-binding domain (RBD) of the SARS-CoV-2 spike (S) protein.

12. The composition of claim 11, wherein the SARS-CoV-2 antigen comprises a truncated, recombinant S protein devoid of signal peptide, transmembrane, and cytoplasmic domains of a full length S protein.

13. The composition of claim 3, wherein the SARS-CoV-2 antigen further comprises one or more of the SARS-CoV-2 membrane (M) protein, nucleocapsid (N) protein, and envelope (E) protein.

14. The composition of any one of claims 1-13, further comprising an aluminum salt adjuvant.

15. The composition of claim 14, wherein the aluminum salt adjuvant comprises one or more of the group consisting of amorphous aluminum hydroxyphosphate sulfate, aluminum hydroxide, aluminum phosphate, and potassium aluminum sulfate.

16. The composition of claim 14, wherein the aluminum salt adjuvant comprises aluminum hydroxide.

17. The composition of claim 15, wherein a 0.5 ml dose of the immunogenic composition comprises from about 0.25 to about 0.50 mg Al³⁺.

18. The composition of claim 17, wherein the mammalian subject is a human subject.

19. A kit comprising:
i) the immunogenic composition of claim 14, and
ii) instructions for administration of the composition to stimulate an immune response against the SARS-CoV-2 antigen in the mammalian subject.

20. The kit of claim 19, further comprising iii) a syringe and needle for intramuscular injection of the immunogenic composition.

21. A method for stimulating an immune response against a severe acute respiratory syndrome coronavirus 2 (SARS-CoV-2) in a mammalian subject, comprising administering the immunogenic composition of claim 14 to a mammalian subject so as to stimulate an immune response against the SARS-CoV-2 antigen in the mammalian subject.

22. The method of claim 21, wherein the mammalian subject is a human subject and/or the immunogenic composition is administered by intramuscular injection.

23. Use of the immunogenic composition of claim 14 for stimulating an immune response against a severe acute respiratory syndrome coronavirus 2 (SARS-CoV-2) in a mammalian subject, the method comprising administering to the subject an effective amount of the immunogenic composition.

24. Use of the immunogenic composition of claim 14 for protecting a mammalian subject from infection with severe acute respiratory syndrome coronavirus 2 (SARS-CoV-2), the method comprising administering to the subject an effective amount of the immunogenic composition.

25. Use of the immunogenic composition of claim 14 for preventing a mammalian subject from contracting COVID-19 disease, the method comprising administering to the subject an effective amount of the immunogenic composition.

26. The use of any one of claims 23-25, wherein the mammalian subject is a human subject and/or the immunogenic composition is administered by intramuscular injection.


SEQUENCE LISTING (5 sequences)

SEQ ID NO: 1 - DNA, 22 bp, Artificial Sequence
  Synthetic Construct
tgactgtgaacgttcgagatga

SEQ ID NO: 2 - DNA, 11 bp, Artificial Sequence
  Synthetic Construct
  misc_feature 3, 7, 11: 2'-deoxy-7-deazaguanosine
tcgaacgttcg

SEQ ID NO: 3 - DNA, 10 bp, Artificial Sequence
  Synthetic Construct
aacgttcgag

SEQ ID NO: 4 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 73 aa, SARS-CoV-2
NSNNLDSKVGGNYNYLYRLFRKSNLKPFERDISTEIYQAGSTPCNGVEGFNCYFPLQSYGFQPTNGVGYQPYR